(19) United States
(12) Patent Application Publication
(10) Pub. No.: US 2007/0161609 A1
(43) Pub. Date: Jul. 12, 2007

(54) Use of mitochondrially targeted antioxidant in treatment and prevention of drug-induced liver disease

(76) Inventors: Charles Buck, West Lafayette, IN (US); Helmut Denk, Graz (AT); Eleonore Frohlich, Graz (AT); Birgit Gutmann, Graz (AT); Peter Hecht, Graz (AT); Ivica Kvietikova, Graz (AT); Marcus Otte, Graz (AT); Gottfried Schatz, Reinach (CH); Cornelia Stumptner, Graz (AT); Lionel Wightman, Vienna (AT); Kurt Zatloukal, Graz (AT)

Correspondence Address: WENDEROTH, LIND & PONACK, L.L.P., 2033 K STREET N. W., SUITE 800, WASHINGTON, DC, 20006-1021, US

(21) Appl. No.: 11/328,247

(22) Filed: Jan. 10, 2006

(51) Int. Cl. A61K 31/66; A61K 31/41

(52) U.S. Cl. 514/130; 514/359

(57) ABSTRACT

The present invention relates to the method of treatment or prophylaxis of patients with drug-induced liver diseases which comprises administering to the patient in need thereof a therapeutically effective amount of a mitochondrially targeted antioxidant compound e.g. derivatives of vitamin E, coenzyme Q₁₀ or a glutathione peroxidase mimetic. The present invention also relates to pharmaceutical compositions containing the antioxidant(s) intended for such use.

### TECHNICAL FIELD

The present invention relates to a method of treating patients with drug-induced liver diseases using a mitochondrially targeted antioxidant, e.g. derivatives of vitamin E, coenzyme Q₁₀ or a glutathione peroxidase mimetic.

### BACKGROUND ART

The liver is the principal organ that is capable of converting drugs into forms that can be readily eliminated from the body. Given the diversity of drugs in use today and the extra metabolic burden they impose upon the liver, it is not surprising that a broad spectrum of adverse drug's effects on liver functions and structures has been documented.

A very large number of drugs used as medicaments for treatment of the human body have been implicated as potential cause of drug-induced liver disease (DILD), but with variable risk of both frequency and severity. These drugs can cause damage to the liver in a variety of ways ranging from mild and transient changes of liver function to complete liver failure with death of the host. Estimates vary widely but e.g. 25-50% of all cases of hepatitis failure (representing the most common drug-induced liver disorder) may be due to adverse drug affects (www.patient.co.uk/showdoc/40001905/, Aug. 15, 2005). Overall, drugs are the most common cause of fulminant hepatic failure, both in the United States and Europe (Björnsson, E. and Olsson, R., 2005, Hepatology, 42, 2:481-489).

Symptoms of drug-induced liver damage are very similar or identical to other causes of liver damage. Thus identifying e.g. drug-induced hepatitis relies on the history of exposure more than on any particular finding on examination or investigation. Clinical evidence of sensitivity to a medication may occur on the first day of its use or only several months later, depending on the medication. Usually, the onset is abrupt, with chills, fever, rash, pruritus, arthralgia, headache, abdominal pain, anorexia, nausea and vomiting. Later, overt evidence of liver damage such as jaundice, dark urine and an enlarged and tender liver may develop. Two general pathogenic mechanisms are recognized: (a) predictable or direct: usually follows promptly an exposure to a new medication. The mechanism appears to be due to direct toxicity or a toxic metabolite, e.g. paracetamol (b) unpredictable or idiosyncratic: may be related to immune hypersensitivity; rash, fewer and eosinophilia is typically present. These reactions follow exposure for a few weeks by e.g. amoxicillin, clavulanic acid. Late onset idiosyncratic reactions are difficult to recognize. They follow exposure for many months and usually do not display features of hypersensitivity, observed e.g. with isoniazid (www.patient.co.uk/showdoc/40001905/, Aug. 15, 2005). The prognosis for patients with acute liver failure due to idiosyncratic drug reactions is usually poor, with 60% to 80% mortality without liver transplantation (Hoofnagle, J. H. et al., 1995, Hepatology, 21:240-252).

Drug-induced liver disease typically presents one of three clinical patterns (a) hepatitis/steatohepatitis: elevated aspartate amino transferase/alanine amino transferase (AST/ALT) (caused e.g. by paracetamol, thiazolidinediones, statins), (b) cholestasis: elevated alkaline phosphatase (caused e.g. by chlorpromazine, erythromycin, oestrogens), (c) mixed picture with damage to both biliary canaliculi and hepatocytes: variable elevations in aminotransferases and alkaline phospahtase (caused e.g. by augmentin).

The combination of high serum transferases (hepatocellular injury) and jaundice has in earlier studies been reported to result in mortality of 10% to 50% for different drugs used as medicaments (Björnsson, E. and Olsson, R., 2005, Hepatology, 42, 2: 481-489). These observations have been named “Hy's rule” after Hyman Zimmerman, who first described them. The rule states that if both drug-induced hepatocellular injury and jaundice occur at the same time without biliary obstruction, mortality of at least 10% can be expected (Björnsson, E. and Olsson, R., 2005, Hepatology, 42, 2: 481-489). Hy's rule has been advocated by the U.S: Food and Drug Administration for use in the assessment of the hepatoxicity of newly developed drugs (Björnsson, E. and Olsson, R., 2005, Hepatology, 42, 2: 481-489).

Many drugs used as medicaments for treatment and/or prophylaxis of the human body may affect the liver adversely in more than one way, and it is not possible to list all adverse effects since new drugs are always being approved for general use. A general list of liver damage induced by drug(s) comprises (a) acute hepatocellular damage either dose-unrelated, e.g. due to antituberculous drugs, halothane, and anticonvulsants, or dose-related, e.g. due to paracetamol (acetaminophen), amiodarone, and methotrexate; (b) chronic active hepatitis, e.g. due to isoniazid and nitrofurantoin; (c) cirrhosis, e.g. due to methotrexate; (d) hepatic tumours, e.g. due to anabolic steroids and combined oral contraceptives; (e) intrahepatic cholestasis: either dose-unrelated, e.g. due to carbimazole, erythromycin, and phenothiazines, or dose-related, e.g. due to anabolic steroids, azathioprine, and oestrogens, such as tamoxifen; (f) other liver toxicity, e.g. induced by statins, HIV drugs, such as neviparin, ritonavir, atazanavir, indinavir, efavirenz, nelfinavir and various nucleoside analogues, and diet agents such as lipokinetix; (g) gallstones, e.g. due to clofibrate and oestrogens.

There is generally no applicable treatment available for drug-induced hepatitis other than discontinuing the medication that is causing the problem. Unfortunately, other than the use of N-acetylcysteine for paracetamol hepatotoxicity, there are no specific antidotes for drug-induced liver disease. Supportive care for acute liver failure and even liver transplantation may be required (http://www.patient.co.uk/showdoc/40001905/, Aug. 15, 2005).

In case of drug-induced steatoheaptitis (caused by four main drugs such as tamoxifen, di-ethylaminoethoxyhexestrol, perhexiline or amiodarone) the enhanced mitochondrial reactive oxygen species (ROS) formation has been suggested to play a major role in the development of steatohepatitis lesions (Pessayre, D. et al. 2001, Seminars in liver diseases, 21, 1: 57-69). Furthermore, the oxidative stress having impact on liver damage (procancero-genic effect) during the chemotherapy event (e.g cyclosporine; Rezzani, R., 2005, Histol. Histopathol., 3: 301-16) has been implicated. In addition, the synergy of the hepatitis B virus (HBV) with aflatoxin causing inflammation and subsequent oxidative damage is striking—the occurrence of liver cancer increases dramatically in infected people (Executive summary of cancer etiology think tank at http://dcb.nci.nih.gov/thinktank/Executive_Summary_of_Cancer_Etiology_Think_Tank.cf m) Cholestasis has been described as an indirect cause for the increase of oxidative stress (Bomzon, A. et.al., 1997, Semin. Nephrol., 17, 6: 549-62 and Huang, Y. T. et.al., 2003, J.Biomed.Sci, 10, 2: 170-8). There has been performed studies on paracetamol induced liver damage in rats to investigate the antioxidant effect of D003 (a mixture of very high molecular weight aliphatic primary acids purified from sugar cane Saccharum officinarum L) wax. The experiments have shown that D003 not only offers cholesterol lowering and antiplatelet effects, but also protects against plasma lipoprotein oxidation resulting in decrease of turgent cells, inflammatory infiltrates and necrotic hepatocytes (Mendoza, S. et.al.2003, MEDJSIM, 25, 3: 169-74). However, so far there were no reports on the effect of altered antioxidants, namely mitochondrially targeted antioxidants, on the enhanced formation of reactive oxygen species (ROS) in this cellular compartement.

It is already known that various antioxidants could be targeted to mitochondria by their covalent attachment to lipophilic cations by means of an alkylene chain (Smith R. A. J. et al., 1999, Eur. J. Biochem., 263: 709-716; Kelso G. F. et al., 2001, J. Biol. Chem., 276: 4588-4596; James A. M. et al., 2005, J. Biol. Chem, 280: 21295-21312). This approach allows antioxidants to be targeted to a primary production site of free radicals and reactive oxygen species within the cell, rather than being randomly dispersed.

In particular, the targeting of vitamin E and coenzyme Q₁₀ derivatives (U.S. Pat. No. 6,331,532; WO 99/26954; EP 1047701; WO2005/016322 and WO2005/016323) or a glutathione peroxidase mimetic (WO 2004/014927) to mitochondria by linking them to the triphenyl phosphonium ion has been described. Experiments in vitro showed that [2-(3,4-dihydro-6-hydroxy-2,5,7,8-tetramethyl-2H-1-benzopyran-2-yl)ethyl]-triphenylphosphonium bromide (MitoVit E) and a mixture of MitoQuinol [10-(3,6-dihydroxy-4,5-dimethoxy-2-methylphenyl)decyl]triphenylphosphonium bromide and MitoQuinone [10-(4,5-dimethoxy-2-methyl-3,6-dioxo-1,4-cyclohexadien-1-yl)decyl]triphenylphosphonium bromide (MitoQ) (Kelso G. F. et al., loc. cit., and Smith R. A. J et al., loc. cit.) or a MitoQ compound wherein the anion is methanesulfonate (James A. M. et al., 2005, J. Biol. Chem, 280: 21295-21312; WO2005/016322 and WO2005/016323) are rapidly and selectively accumulated by mitochondria within isolated cells.

In addition, a mitochondria-targeted derivative of the spin trap phenyl-t-butylnitrone (Mi-toPBN) has been developed (Smith R. A. J., Bioenergetics Group Colloquium, 2003, 679^{th} Meeting of the Biochemical Society: 1295-1299).

Importantly, the accumulation of these antioxidants by mitochondria protected them from oxidative damage far more effectively than untargeted antioxidants, suggesting that the accumulation of bioactive molecules within mitochondria does increase their efficacy while also decreasing harmful side reactions (Murphy M. P. and Smith R. A. J., 2000, Adv. Drug. Delivery Rev., 41: 235-250).

Furthermore, it was found that the simple alkyltriphenylmethylphosphonium cation (TPMP), MitoVit E and MitoQ could be fed safely to mice on a long term basis, generating potentially therapeutically effective concentrations within the brain, heart, liver, and muscle (Smith R. A. et al., 2003, PNAS, 100(9): 5407-5412).

Application of these compounds (U.S. Pat. No. 6,331,532, WO 99/26954, EP 1047701; WO 2004/014927, WO2005/016322 and WO2005/016323) was claimed for use in preventing the elevated mitochondrial oxidative stress associated with neurodegenerative diseases, such as Parkinson's disease, Friedrich's Ataxia, Wilson's disease, diseases associated with mitochondrial DNA mutations, diabetes, motor neuron disease, inflammation and ischemic reperfusion tissue injury in strokes, heart attacks, organ transplantation and surgery, and the non-specific loss of vigour associated with ageing. In addition use of these compounds as prophylactics to protect organs during transplantation, to ameliorate the ischemic reperfusion injury that occurs during surgery, to reduce cell damage following stroke and heart attack, or as prophylactics given to premature babies, who are susceptible to brain ischemia, has been claimed in the mentioned patent documents.

### SUMMARY OF THE INVENTION

The invention relates to a method of treating patients with drug-induced liver diseases using a mitochondrially targeted antioxidant compound comprising a lipophilic cation covalently coupled to an antioxidant moiety.

### DETAILED DESCRIPTION OF THE INVENTION

It has now unexpectedly been found that mitochondrially targeted antioxidants, e.g. derivatives of vitamin E, coenzyme Q₁₀ or glutathione peroxide mimetic, are useful in the treatment and/or prevention of drug-induced liver diseases.

In its broadest aspect, the invention provides a method of treating patients with drug-induced liver diseases using a mitochondrially targeted antioxidant which comprises a lipophilic cation covalently coupled to an antioxidant moiety, wherein the antioxidant moiety is capable of being transported through the mitochondrial membrane and accumulated within the mitochondria of intact cells. In particular, the compound according to invention prevents cellular damage resulting from oxidative stress (or free radicals) in the mitochondria.

A term “drug-induced liver disease” according to invention refers to and comprises all kinds of disorders induced by drugs that affect the anatomy, physiology, metabolism, and/or genetic activities of the liver, that affect the generation of new liver cells and/or the regeneration of the liver, as a whole or parts thereof, transiently, temporarily, chronically or permanently, in a pathological way.

The term “drug-induced liver disease” according to invention refers to and comprises diseases with pathological conditions such as (a) acute dose-dependent- or acute dose-independent liver damage (causing e.g. acute hepatitis, cholestatic jaundice, acute fatty infiltration of the liver) and/or damage to liver blood vessels (b) chronic liver disease (causing e.g. chronic hepatitis, liver cirrhosis or fibrosis, chronic cholestasis) (c) chronic inflammatory nodules (causing e.g. liver granulomas) (g) hyperproliferative diseases, e.g. liver tumors (benign and malignant).

The term “drug” according to the invention comprises but not limited to all kinds of drugs for (a) oncology treatment (preferably in liver cancer treatment), such as chemostatic drugs, e.g. tamoxifen (b) preventive medication (such as pentoxifilin) or herbal and dietary supplements used alone or in combination thereof (e.g. echinacea, ginkgo, garlic, ginger, bilberry, dong quai, feverfew, ginseng, turmeric, meadowsweet and willow), or used in combination with other drugs inducing liver disease(s) such as analgesic drugs (e.g. aspirin, acetaminophen) (c) antiviral treatment, such as anti HIV drugs, e.g. ritonavir, or antibiotic treatment, such as sulfonamid; particularly for people co-infected with hepatitis C (d) similar treatment of chronic viral infections such as hepatitis B and C with e.g. interferon, ribavirin. Furthermore, the co-taking of alcohol and many drug combinations used for colds (e.g. acetaminophen which is an ingredient in some over-the counter pain relievers (e) treatment of diabetes (e.g. insulin sensitizers) (f) other non cancer treatment, such as antinflammatory drugs (e.g. paracetamol), antiarrhythmica (e.g. amiodarone), anticonvulsant (antiepileptic) drugs (e.g. valproate and phenyloin), H2 blockers (e.g. ranitidine), anti-depressive (e.g. cymbalta), steroids (such as glucocorticoids, anabolics), statins (e.g. pravastin), methyldopa used uncommonly for high blood pressure, chlorpromazine, amiodarone used for irregular heart rhythm, certain antibiotics (including isoniazide for tuberculosis, trimethoprim-sulfamethoxazole and erythromycin), anesthetics (e.g. halothane), resulting in drug-induced liver disease(s).

The term “drug” according to the invention refers to and comprises all nonsteroidal antiinflamatory drugs, anticancer drugs, antibiotics, anticonvulsants, antiviral drugs, tuberculostatics, anesthetics, analgetics, drugs for treatment of cardiovascular diseases, antidepressiva, steroids, statins, insulin sensitizers, H2 blockers, herbal and dietary supplements or other drugs inducing a mild, severe, acute or chronic liver disease

Since there is a daily update of the approved drugs, there is no possibility to list all medicaments/prophylactics inducing liver diseases. A representative (but not the exhaustive) list of drugs according to the invention available on the market comprises: acetaminophen (paracetamol), salicylates, acebutolol, indomethacin, phenylbutazone, allolpurinol, isoniazid, phenyloin, atenolol, ketoconazole, piroxicam, carbamapezine, labetalol, probenecid, cimitidine, maprotiline, pyrazinamide, dantrolene, metoprolol, quinidine, diclofenac, mianserin, quinine, ditiazem, naproxen, rantidine, enflurane, para-aminosalicylic acid, sulfonamides, ethambutol, penicillins, sulindac, ethionamide, phenelzine, tricyclic antidepressants, halothane, phenindione, valproic acid, ibuprofen, Phenobarbital, verapamil, adrenocortical steroids, phenothizines, sulfonamides, antithyroid drugs, phenotoin, tetracyclines, isoniazid, salicylates, valproic acid, methotrexate, actinomycin D, chlorpropamide, erythromycin, amoxicillin/clavulanate, carbamazepine, danazol, glyburide, carbimazole, diazepam, enalapril, haloperidol, ketoconazole, norethandrolone, sulfonamides, nifedipine, penicillamine, tolbutamide, nitrofurantoin, phenothiazines, cloxacillin flecainide, azathioprine, cyclophosphamide, flurazepam, captopril, cyclosporine, flutamide, gold, cephalossporines, disopyramide, griseofulvin, chlordiazepoxide, enalapril, mercaptopurine, oral contraceptives, tamoxifen, methyltestosterone, oxacillin, thiazobendazole, tricyclic antipdepressants, nonsteroidal, phenyloin, troleandomycin, anti-inflammatory drugs, propoxyphene, verapamil, allopurinol, phenyloin, aspirin, hydralazine, procainamide, carbamazepine, isoniazid, quinidine, chlorpromazine, isoniazid, quinidine, nitrofurantoin, diltiazem, tolbutamide, disopyramide, phenylbutazone, dantrolene, methyldopa, isoniazid, nitrofurantoin, terbinafine HCl (lamisil, sporanox), nicotinic acid, chlorpromazine/valproic acid combination, imipramine, thiabendazole, phenothiazines, tolbutamide, chloropropamide/erythromycin combination, phenyloin, anabolic steroids, thorotrast, danazol, testosterone, adriamycin, dacarbazine, thioquanine, mercaptopurine, vincristine, azathioprine, vitamin A, carmustine, mitomycin, cyclophospoamide/cyclosporine combination, glucocorticoids, rifampin, pyrazinamide, prednisolone, fluvastatin, pravastin.

Furthermore, this list encompasses also commonly used herbal and dietary supplements, (e.g. those that are known to possess antiplatelet activity such as ginkgo, garlic, ginger, bilberry, dong quai, feverfew, ginseng, turmeric), herbs containing coumarin (e.g.chamomile, motherworth, horse chestnut, fenugreek and red clover), other potentially hepatotoxic herbs (preferably echinacea and kava), herbs containing salicylate (e.g. willow, meadowsweet), sedative herbal supplements (such as valerian), tamarind, kyushin, licorice, plantain, uzara root, hawthorn, karela, evening primrose oil, borage, St John wort, saw palmetto, shankapulshpi, kelp, used alone or in the combination thereof, or in combination with other drugs inducing liver disease(s) according to the invention, preferably with analgesic drugs, non-steroidal anti-inflammatory drugs (NSAIDs), particularly aspirin or acetaminophen, anabolic steroids, amiodarone, methotrexate, ketoconazole, opioid analgesics, anticonvulsants, barbiturates, immunosuppressants (eg, corticosteroids and cyclosporine), drugs used in thyroid replacement therapies, digoxin, phenyloin, spironolactone, drugs used for diabetes treatment.

The invention relates to the method of treatment or prophylaxis of patients with drug-induced liver diseases which comprises administering to the patient in need thereof a therapeutically effective amount of a mitochondrially targeted antioxidant compound comprising a lipophilic cation covalently coupled to an antioxidant moiety.

A preferred embodiment represents the method of treatment or prophylaxis of patients with drug-induced liver diseases which comprises administering to the patient in need thereof a therapeutically effective amount of a mitochondrially targeted antioxidant compound comprising a lipophilic cation covalently coupled to an antioxidant moiety, wherein the drug-induced liver disease is a disease with pathological condition selected from the group consisting of acute liver damage, damage to liver blood vessels, chronic liver damage, chronic inflammatory nodules and hyperproliferation in the liver.

Even more preferred embodiment represents the method of treatment or prophylaxis of patients with drug-induced liver diseases which comprises administering to the patient in need thereof a therapeutically effective amount of a mitochondrially targeted antioxidant compound comprising a lipophilic cation covalently coupled to an antioxidant moiety, wherein the acute liver damage is a dose-dependent or dose-independent liver damage.

Yet another preferred embodiment is the method of treatment or prophylaxis of patients with drug-induced liver diseases which comprises administering to the patient in need thereof a therapeutically effective amount of a mitochondrially targeted antioxidant compound comprising a lipophilic cation covalently coupled to an antioxidant moiety, wherein the disease is a disease selected from the group consisting of acute hepatitis, cholestatic jaundice, acute fatty infiltration of the liver, chronic hepatitis, liver cirrhosis, liver fibrosis, chronic cholestasis, liver granulomas and liver tumours (benign and malignant).

Still more preferred embodiment is the method of treatment or prophylaxis of patients with drug-induced liver diseases which comprises administering to the patient in need thereof a therapeutically effective amount of a mitochondrially targeted antioxidant compound comprising a lipophilic cation covalently coupled to an antioxidant moiety, wherein the disease is the acute hepatitis or cholestatic jaundice.

A further preferred embodiment represents the method of treatment or prophylaxis of patients with drug-induced liver diseases which comprises administering to the patient in need thereof a therapeutically effective amount of a mitochondrially targeted antioxidant compound comprising a lipophilic cation covalently coupled to an antioxidant moiety-wherein the drug inducing liver disease(s) is a drug from the group consisting of nonsteroidal antiinflamatory drugs, anticancer drugs, antibiotics, anticonvulsants, antiviral drugs, tuberculostatics, anesthetics, analgetics, antiarrythmics, antidepressiva, steroids (such as glucocorticoids, anabolics), statins, insulin sensitizers, antihistamnica and herbal and dietary supplements.

Yet another preferred embodiment is the method of treatment or prophylaxis of patients with drug-induced liver diseases which comprises administering to the patient in need thereof a therapeutically effective amount of a mitochondrially targeted antioxidant compound comprising a lipophilic cation covalently coupled to an antioxidant moiety, wherein the drug is a nonsteroidal antiinflamatory- or an anticancer drug.

Still another preferred embodiment is the use of a mitochondrially targeted antioxidant compound comprising a lipophilic cation covalently coupled to an antioxidant moiety for the treatment or prophylaxis of drug-induced liver diseases.

Within the meaning of the invention the term “disease according to invention” encompasses drug-induced liver disorders as defined above.

A preferred embodiment represents the method of treatment or prophylaxis of patients with drug-induced liver diseases which comprises administering to the patient in need thereof a therapeutically effective amount of a mitochondrially targeted antioxidant compound comprising a lipophilic cation covalently coupled to an antioxidant moiety wherein the liphophilic cation is the triphenylphosphonium cation.

Other lipophilic cations which may covalently be coupled to antioxidants in accordance with the present invention include the tribenzyl or triphenyl ammonium cation or the tribenzyl or a substituted triphenyl phosphonium cation.

In another preferred embodiment said mitochondrially targeted compound according to invention has the formula P(Ph)₃⁺XR·Z⁻ wherein X is a linking group, Z⁻ is an anion and R is an antioxidant moiety and the lipophilic cation represents the triphenylphosphonium cation, as shown by the general formula 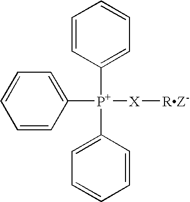
X as a linking group may be a carbon chain, one or more carbon rings, or a combination thereof, and such chains or rings wherein one or more carbon atoms are replaced by oxygen (forming ethers or esters) and/or by nitrogen (forming amines or amides).

While it is generally preferred that the carbon chain is an alkylene group, carbon chains which include one or more double or triple bonds are also within the scope of the invention. Also included are carbon chains carrying one or more substituents (such as oxo, hydroxyl, carboxylic acid or carboxamide groups), and/or one or more side chains or branches selected from unsubstituted or substituted alkyl, alkenyl or alkynyl groups.

Preferably, X is a C₁-C₃₀, more preferably C₁-C₂₀, most preferably C₁-C₁₅ carbon chain.

Preferably, X is (CH₂)ₙ, wherein n is an integer from 1 to 20, more preferably from about 1 to about 15.

In some particularly preferred embodiments, the linking group X is an ethylene, propylene, butylene, pentylene or decylene group.

In one particularly preferred embodiment the antioxidant moiety R is a quinone. In another preferred embodiment the antioxidant R moiety is a quinol. A quinone and corresponding quinol are equivalents since they are transformed to each other by reduction and oxidation, respectively.

In other embodiment the antioxidant moiety R is selected from the group consisting of vitamin E and vitamin E derivatives, chain breaking antioxidants, including butylated hydroxyanisole, butylated hydroxytoulene, general radical scavengers including derivatised fullerenes, spin traps including derivatives of 5,5-dimethylpyrroline N-oxide, tert-butylnitrosobenzene, a-phenyl-tert-butylnitrone and related compounds.

In a further preferred embodiment the antioxidant moiety R is vitamin E or a vitamin E derivative.

In another preferred embodiment the antioxidant moiety R is butylated hydroxyanisole or butylated hydroxytoulene.

In still further preferred embodiment the antioxidant moiety R represents a derivatised fullerene.

In some particularly preferred embodiments the antioxidant moiety R is a 5,5-dimethylpyrroline N-oxide, tert-butylnitrosobenzene, a-phenyl-tert-butylnitrone and derivatives thereof.

Preferably, Z⁻ is a pharmaceutically acceptable anion. Such pharmaceutically acceptable anions are formed from organic or inorganic acids. Suitable inorganic acids are, for example, halogen acids, such as hydrochloric acid, hydrobromic acid, sulfuric acid, or phosphoric acid. Suitable organic acids are, for example, carboxylic, phosphonic, sulfonic or sulfamic acids, for example acetic acid, propionic acid, octanoic acid, decanoic acid, dodecanoic acid, glycolic acid, lactic acid, fumaric acid, succinic acid, adipic acid, pimelic acid, suberic acid, azelaic acid, malic acid, tartaric acid, citric acid, amino acids, such as glutamic acid or aspartic acid, maleic acid, hydroxymaleic acid, methylmaleic acid, cyclohex-anecarboxylic acid, adamantanecarboxylic acid, benzoic acid, salicylic acid, 4-aminosalicylic acid, phthalic acid, phenylacetic acid, mandelic acid, cinnamic acid, alkane sulfonic acid such as methane- or ethane-sulfonic acid, 2-hydroxyethanesulfonic acid, ethane-1,2-disulfonic acid, arylsulfonic acid such as benzenesulfonic acid, 2-naphthalenesulfonic acid, 1,5-naphthalene-disulfonic acid or 2-, 3- or 4-methylbenzenesulfonic acid, methylsulfuric acid, ethylsulfuric acid, dodecylsulfuric acid, N-cyclohexylsulfamic acid, N-methyl-, N-ethyl- or N-propyl-sulfamic acid, or other organic protonic acids, such as ascorbic acid.

In one preferred embodiment Z⁻ is halide. In another preferred embodiment Z⁻ is bromide.

In a further preferred embodiment Z⁻ is the anion of an alkane- or arylsulfonic acid. In one particularly preferred embodiment Z⁻ is methanesulfonate.

In another particularly preferred embodiment, the mitochondrially targeted antioxidant useful in the treatment and prevention of liver diseases and/or epithelial cancers has the formula 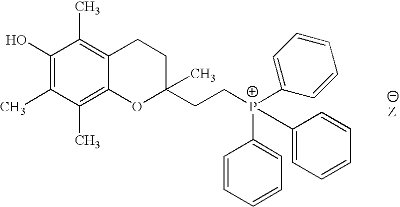
including all stereoisomers thereof wherein Z⁻ is a pharmaceutically acceptable anion, preferably Br⁻. This compound is referred to herein as “MitoVit E”.

In another preferred embodiment, the mitochondrially targeted antioxidant useful in the treatment and prevention of diseases according to the invention has the general formula 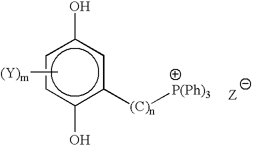
wherein Z⁻ is a pharmaceutically acceptable anion, preferably a halogen, m is an integer from 0 to 3, each Y is independently selected from groups, chains and aliphatic and aromatic rings having electron donating and accepting properties, (C)ₙ represents a carbon chain optionally carrying one or more double or triple bonds and optionally including one or more substituents and/or unsubstituted or substituted alkyl, alkenyl or alkynyl side chains, and n is an integer from 1 to 20.

Preferably, each Y is independently selected from the group consisting of alkoxy, alkylthio, alkyl, haloalkyl, halo, amino, nitro, optionally substituted aryl, or when m is 2 or 3, two Y groups, together with the carbon atoms to which they are attached, form an aliphatic or aromatic carbocyclic or heterocyclic ring fused to the aryl ring. More preferably, each Y is independently selected from methoxy and methyl.

Preferably, (C)ₙ is an alkyl chain of the formula (CH₂)ₙ.

In a particularly preferred embodiment, the mitochondrially targeted antioxidant according to the invention has the formula 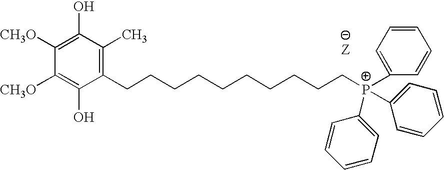
wherein Z⁻ is a pharmaceutically acceptable anion, preferably Br⁻ referred to herein as “MitoQuinol”, or an oxidized form of the compound (wherein the hydroquinone of the formula is a quinone) referred to herein as “MitoQuinone”. A mixture of varying amounts of MitoQuinol and MitoQuinone is referred to as “MitoQ”.

Even more preferably, the mitochondrially targeted antioxidant according to the invention has the formula 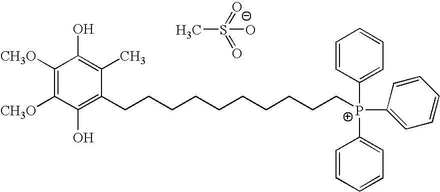
wherein the pharmaceutically acceptable anion Z⁻ is methanesulfonate. In this embodiment a mixture of varying amounts of MitoQuinol and MitoQuinone is referred to as “MitoS”.

Further preferred embodiment according to invention represents the mitochondrially targeted derivative of the spin trap phenyl-t-butylnitrone of the following formula 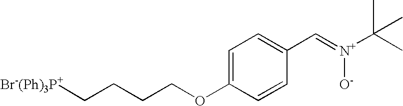
referred to herein as “MitoPBN”.

In another embodiment according to the invention the mitochondrially targeted antioxidant is a glutathione peroxidase mimetic such as a selenoorganic compound, i.e. an organic compound comprising at least one selenium atom. Preferred classes of selenoorganic glutathione peroxidase mimetics include benzisoselenazolones, diaryl diselenides and diaryl selenides.

In particular the glutathione peroxidase mimetic moiety is 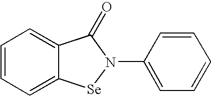
referred to herein as “Ebelsen” (2-phenyl-benzo[d]isoselenazol-3-one).

Preferred compounds of the invention have the formula 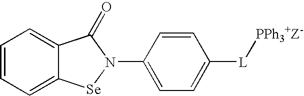
wherein Z⁻ is a pharmaceutically acceptable anion, preferably Br⁻ and L is a monosaccharide.

One particularly preferred embodiment according to invention has the formula 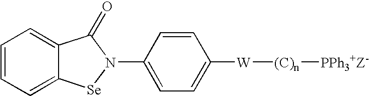
wherein Z⁻ and (C)ₙ are defined as above, W is O, S or NH, preferably O or S, and n is from 1 to 20, more preferably 3 to 6.

In a further aspect, the present invention provides a pharmaceutical composition suitable for treatment and/or prophylaxis of a patient suffering from drug-induced liver disease and/or epithelial cancer, which comprises an effective amount of a mitochondrially targeted antioxidant according to the present invention in combination with one or more pharmaceutically acceptable carriers or diluents, such as, for example, physiological saline solution, demineralized water, stabilizers (such as β-cyclodextrin, preferably in ratio 1:2), and/or proteinase inhibitors.

The term “pharmaceutically acceptable” as used herein pertains to compounds, ingredients, materials, compositions, dosage, forms etc., which are within the scope of sound medical judgment, suitable for use in contact with the tissues of the subject in question (preferably human) without excessive toxicity, irritation, allergic response, or other problem or complication, commensurate with a reasonable benefit/risk ratio. Each carrier, diluent, excipient etc. must also be “acceptable” in the sense of being compatible with the other ingredients of the formulation.

In still a further aspect, the invention provides a method of treatment or prophylaxis of a patient suffering from drug-induced liver disease who would benefit from reduced oxidative stress, which comprises the step of administering to said patient a mitochondrially targeted antioxidant as defined above.

The term “treatment” within the meaning of the invention refers to a treatment that preferably cures the patient from at least one disorder according to the invention and/or that improves the pathological condition of the patient with respect to one or more symptoms associated with the disorder, on a transient, short-term (in the order of hours to days), long-term (in the order of weeks, months or years) or permanent basis, wherein the improvement of the pathological condition may be constant, increasing, decreasing, continuously changing or oscillatory in magnitude as long as the overall effect is a significant improvement of the symptoms compared with a control patient.

Further, the term “treatment” as used herein in the context of treating liver diseases pertains generally to treatment and therapy of a human in which some desired therapeutic effect is achieved, for example the inhibition of the progress of the condition, and includes a reduction in the rate of progress, a halt in the rate of progress, amelioration of the condition, and cure of the condition.

The term “treatment” according the invention includes combination treatments and therapies, in which two or more treatments or therapies are combined, for example sequentially or simultaneously. Treatment as a prophylactic measure (i.e. prophylaxis) is also included.

Prophylaxis refers to any medical or public health procedure whose purpose is to prevent, rather than treat or cure, disease(s) according to the invention. Roughly, prophylactic measures are divided between primary prophylaxis (to prevent the development of a disease according the invention) and secondary prophylaxis (where the disease has already developed and the patient is protected against worsening of this process).

Treatment according to the invention can be carried out in a conventional manner generally known to the person skilled in the art, e.g. by means of oral application or via intravenous injection of the pharmaceutical compositions according to the invention.

Therapeutic efficacy and toxicity, e.g. ED₅₀ and LD₅₀, may be determined by standard pharmacological procedures in cell cultures or experimental animals. The dose ratio between therapeutic and toxic effects is the therapeutic index and may be expressed by the ratio LD₅₀/ED₅₀. Pharmaceutical compositions that exhibit large therapeutic indexes are preferred. The dose must be adjusted to the age, weight and condition of the individual patient to be treated, as well as the route of administration, dosage form and regimen, and the result desired, and the exact dosage should of course be determined by the practitioner.

The actual dosage depends on the nature and severity of the disorder being treated, and is within the discretion of the physician, and may be varied by titration of the dosage to the particular circumstances of this invention to produce the desired therapeutic effect. However, it is presently contemplated, that pharmaceutical compositions comprising of from about 0.1 to 500 mg/kg of the active ingredient per individual dose, preferably of from about 0.1 to 100 mg/kg, most preferred from about 0.1 to 10 mg/kg, are suitable for therapeutic treatments.

In the method of treating patients as a prophylaxis against drug-induced liver diseases which comprises administering to the patient in need thereof a prophylactically effective amount of a mitochondrially targeted antioxidant compound comprising a lipophilic cation covalently coupled to an antioxidant moiety, the mitochondrially targeted antioxidant may be administered together (separately or in a fixed combination) or in sequence with the drug suspected to cause drug-induced liver disease or inducing liver disease(s) according to the invention.

In general, a suitable dose of the active compound according to invention is in the range of about 0.1 mg to about 250 mg per kilogram body weight of the subject to be treated per day.

The active ingredient may be administered in one or several dosages per day. A satisfactory result can, in certain instances, be obtained at a dosage as low as 0.1 mg/kg intravenously (i.v.) and 1 mg/kg perorally (p.o.). Preferred ranges are from 0.1 mg/kg/day to about 10 mg/kg/day i.v. and from 1 mg/kg/day to about 100 mg/kg/day p.o.

Furthermore the invention relates to the manufacture of medicaments containing the antioxidant compounds according to invention useful in the treatment and/or prevention of drug-induced liver diseases, using standard procedures known in the prior art of mixing or dissolving the active compound with suitable pharmaceutical carriers. Such methods include the step of bringing into association the active compound with a carrier which comprises one or more accessory ingredients. In general the formulations according to invention are prepared by uniformly and intimately bringing into association the active compound with carriers (e.g. liquid carriers, finely divided solid carrier) and then shaping the product, if necessary. Suitable carriers, diluents and excipients used in the present invention can be found in standard pharmaceutical texts (see for example Handbook for Pharmaceutical Additives, 2001, 2^{nd} edition, eds. M. Ash and I. Ash).

The antioxidant compounds according to the invention e.g. derivatives of vitamin E, coenzyme Q₁₀ or a glutathione peroxidase mimetic, may be synthesized according to any of the known processes for making those compounds described in e.g. U.S. Pat. No. 6,331,532, WO 99/26954, WO 2004/014927 or WO 2003/016323).

It will be apparent to those skilled in the art that various modifications can be made to the compositions, methods and processes of this invention. Thus, it is intended that the present invention cover such modifications and variations, provided they come within the scope of the appended claims and their equivalents. All publications cited herein are incorporated in their entireties by reference.

To practically assess the impact of mitochondrially targeted antioxidants, e.g. derivatives of vitamin E, coenzyme Q₁₀ or a glutathione peroxidase mimetic, in the treatment and/or prevention of liver diseases induced by drug(s) according to the invention (e.g by paracetamol or by non steroidal antiinflamatory drug such as carprofen in combination with porphyrogenic agent 3,5-diethoxycarbonyl-1,4-dihydrocollidine (DDC) (Denk H. et al., 1975, Lab. Invest.: 773-776; Tsunoo C. et al., 1987, J. Hepatol., 5: 85-97), the presence of morphological alterations in mouse livers such as the degree of hepatocyte damage including but not limited to ballooning of hepatocytes (enlargement of hepatocytes), necrosis, apoptosis occurring mainly around the central vein is evaluated and compared to non targeted antioxidant compound (such as vitamin E) and non treated group of animals. Preferably, the hepatocyte damage in acute intoxication is analysed by routine haematoxylin and eosin staining (Example 3, FIGS. 1a to 1c, 2a/b and 3a/b).

In a further experimental set up to evaluate the oxidative stress in drug-induced liver disorders western blot analysis of hemoxygenase (HO-1) expression level can be employed to analyse the extracts derived from e.g. paracetamol-intoxicated mice (pretreated with MitoQ for 24 hours) with the continuous exposure to paracetamol and simultaneous MitoQ treatment for further 24h. A marked reduction of drug-induced overexpression of the HO-1 (known to be induced by reactive oxygen species (ROS), Suematsu M. and Ishimura Y., 2000. Hepatology, 31(1): 3-6) suggests that oxidative stress is significantly reduced in drug-induced liver disease(s) by antioxidants according to the invention when compared to animals treated with nontargeted antioxidant compound (e.g. vitamin E) or non treated group of animals, respectively (Example 4).

Furthermore, the protein expression level of fatty acid binding protein (FABP) representing a sensitive marker for hepatocyte damage (Monbaliu D. et al., 2005, Transplant Proc., 37(1): 413-416) can be used to show a significant decrease of FABP protein in drug-intoxicated mice (e.g. by using paracetamol) when compared to the control group. Under MitoQ (or MitoS) treatment of this group of animals the FABP protein expression reach almost control mice FABP expression values, thus suggesting again the effect of mitochondrially targeted antioxidants in treatment or prophylaxis of diseases according to the invention (Example 4).

In another experimental set-up to investigate the effect of the mitochodnrially targeted antioxidants (MitoQ/MitoS) in diseases according to invention, serum levels of liver specific enzymes derived from drug-intoxicated versus treated and control mice can be monitored as for example, in the Actitest (Biopredictive, Houilles, France) that provides a measure of liver damage and particularly fibrosis, which is characteristic of several diseases according to the invention. The serum levels of e.g. a₂-macroglobulin, haptoglobin, γ-glutamyl transpeptidase, total bilirubin, apolipoprotein Al and alanine aminotransferase can be measured from paracetamol-intoxicated only, control, and corresponding drug-intoxicated animals simultaneously exposed to the mitochondrially targeted antioxidants using the methods described in Poynard, et al., 2003, Hepatology 38:481-492, by following general time line strategy according to Example 3.

Alternatively, in serum from various tested animal groups following parameters indicating liver damage, namely bilirubin, alanine-aminotransferase (ALT/GPT), aspartate aminotransferase (ASAT/GOT) and glutamate dehydrogenase (GLDH) can be determined according to standard protocols in clinical diagnostics employing commercially available kits (Example 5.). The reduction of serum liver enzymes in the drug-intoxicated animals (as e.g. alanine- and aspartate aminotransferases) treated with the antioxidant compounds according to the invention indicates the reduction of liver damage in such treated samples and provides support for the therapeutic efficacy of these compounds in diseases according to the invention.

To evaluate the production of reactive oxygen species (ROS) one may, for example, employ dihydroethidium (DHE) staining of liver sections (e.g. frozen sections) prepared from control, intoxicated and treated animals according to a standard protocol (Brandes R P et al., Free Radic Biol Med. 2002; 32 (11): 1116-1122). This approach allows demonstration of induction of ROS production in vivo in livers of control-, drug-intoxicated- and treated animals thus mimicking observations made in the patients suffering from the diseases according to the invention (Example 6). Other possibilities to evaluate the ROS formation include e.g. a lucigenin chemiluminescence assay (Goerlach A. et al., 2000, Circ Res., 87(1): 26-32).

The general strategy of timelines and dosage regime(s) for drug-intoxication of tested animals and for their treatment with the antioxidants is identical to the experimental approach used for determination of morphologic abnormalities, according to Example 3.

Chronic liver damage can be assessed in mice intoxicated with valproate (0.71% weight/volume) for up to 21 days when fatty degeneration of hepatocytes, activation of Kupffer cells, inflammation and hepatocyte necrosis can be detected (Raza M. 2000, Int J Tissue React., 22(1): 15-21).

Optionally, in vitro experiments employing immortalized hepatocytes, e.g. by using Fa2N-4 or Ea1C-35 developed by MultiCell Technologies (U.S. Pat. No. 6,107,043) allow measurement of ROS production in liver cells upon drug-intoxication (e.g. by using of paracetamol).

By employing standard protocols according to Example (8), the immortalized hepatocytes can be intoxicated with drugs inducing liver disease(s) according to the invention (concentration range from 1 nM to 1 mM depending on the evaluated substance inducing liver disease) simultaneously treated with MitoQ or MitoS, respectively or vitamin E (in concentrations corresponding to EC₅₀=1 μM for MitoQ and EC₅₀=10 μM for vitamin E) provide a significant reduction in ROS formation, thus further confirming a therapeutic benefit of mitochondrially targeted antioxidants in drug-liver disorders according to the invention.

When compared to the state of the art of therapy or prophylaxis of liver disorders induced by drugs, the method of treatment according to the invention surprisingly provides an improved, sustained and more effective treatment.

The invention will be further illustrated below with the aid of the figures and examples, representing preferred embodiments and features of the invention without the invention being restricted hereto.

### BRIEF DESCRIPTION OF FIGURES

FIG. 1a to 1c: Effect of MitoQ on the degree of hepatocyte damage in mouse liver upon short term (1 day) exposure to paracetamol (500 mg/kg of animal)

Fig 1a: Normal mouse hepatocytes are arranged in strands separated by sinusoids some containing Kuppfer cells (7). The cytoplasm has a granular appearance; inflammatory cells are absent; central vein (5). Magnification 40×.

FIG. 1b: After intoxication with paracetamol (500 mg/kg of animal) for 1 day the sinusoids (1) are filled with blood cells shown originally in red staining, hepatocytes show various degrees of damage ranging from microvesicular steatosis (2) originally white dots, to necrosis (3) originally shown as light pink areas. Inflammatory cells (4) are present throughout the necrotic area; central vein (5). Magnification 40×.

FIG. 1c: The simultaneous treatment with MitoQ (300 nmol/animal dissolved in PBS/1% DMSO) for 1 day (with 24 hours MitoQ pretreatment) reduces blood congestion in the sinusoids (1). Hepatocytes show microvesicular steatosis (2) and only few single necrotic cells are seen (3); central vein (5) Magnification 40×.

FIGS. 2a and 2b: Effect of MitoQ on the degree of hepatocyte damage in mouse liver upon short term (3 days) exposure to carprofen (5 mg/kg in 1% DMS/PBS) in combination with DDC (0.1% in mice diet) FIG. 2a: Upon intoxication with carprofen (5 mg/kg of animal) dissolved in solvent (PBS/1% DMSO) and DDC (0.1%) given within a diet, the morphology of hepatocytes is altered and inflammatory cells (4) are seen in the sinusoids. Enlarged hepatocytes (8) and single necrotic cells are common (3); central vein (5). Magnification 40×.

FIG. 2b: The simultaneous treatment with MitoQ (300 nmol/animal dissolved in PBS/1% DMSO for 3 days restores normal liver morpohology; hepatocytes are arranged in strands and inflammatory cells are absent; central vein (5). Magnification 40×.

FIGS. 3a and 3b: Comparison of MitoQ (12 mg/kg of animal;given peritoneally) and vitamin E (100 mg/kg of animal, applied subcutneously) effect on the degree of hepatocyte damage in mouse liver upon short term (3 days) exposure to DDC (0.1%)

FIG. 3a: The simulatenous treatment with MitoQ restores normal liver morphology; hepatocytes are arranged in strands and inflammatory cells are absent, central vein (5). Protoporphyrin accumulation persists in the bile ducts (9); portal vein (6). Magnification 40×.

FIG. 3b: The simultaneous treatment with vitamin E (100 mg/kg of animal) only partially restores normal liver morphology; hepatocytes are arranged in strands but the inflammatory cells (4) are still present in the periportal area (10). Protoporphyrin accumulation persists in the bile ducts (9); portal vein (6). Magnification 40×.

### EXAMPLES

### Example 1

### Experimental Induction of Hepatocyte Damage in Mouse Liver

Hepatocyte damage in mouse livers by acute or chronic intoxication of various mouse strains: e.g., C57B1/6 mice (Harlan Winkelmann, Germany) with paracetamol (Tylenol, Mc Neil Consumer plus Specially Pharamceuticals Division, US) or non steroidal antiinfalmatory drug carprofen (Rimadyl, Pfizer, US) in combination with 3,5-diethoxycarbonyl-1,4-dihydrocollidine (DDC) (Sigma) is performed.

Animals are kept in conventional cages or in sterile isolators with a 12 hrs day-night cycle. Animals receive humane care according to the criteria outlined in the “Guide for the Care and Use of Laboratory Animals” prepared by the National Academy of Sciences and published by the National Institutes of Health; NIH publication 86-23, revised 1985.

Mice (8 weeks old) are fed a standard diet. Paracetamol is administered by oral gavage (500 mg/kg of animal). Carprofen is given by intraperitoneal injection (three times every 24 hours) in combination with 0.1% DDC which is included in the diet.

After approximately 24 hours of paracetamol- or three days of carprofen- and DDC intoxication respectively, various damage of hepatocytes can be observed ranging from hepatocytes enlargement, microvesicular steatosis to apoptosis and necrosis.

Mice are sacrified at different time-points of intoxication by cervical dislocation and the livers are either immediately snap-frozen in methylbutane precooled with liquid nitrogen for immunofluorescence or fixed in 4% buffered formaldehyde for routine histology and immunohistochemistry.

### Example 2

### Evaluation of Liver Alterations

Liver samples prepared according to Example 1 are used for simple histologic staining such as with haematoxylin and eosin (Luna L. G., 1968, Manual of Histologic staining methods of the Armed Forces Institute of Pathology, 3rd edition. McGraw Hill, New York).

### Example 3

### Effect of the Antioxidants According to the Invention on Liver Pathology

To evaluate the impact of the antioxidants according to the invention on regression of morphological alterations in early stages of paracetamol-intoxicated mice livers, a group of animals are treated on day one with MitoQ (a mixture of MitoQuinol [10-(3,6-dihydroxy-4,5-dimethoxy-2 methylphenyl)decyl]triphenylphosphonium bromide and MitoQuinone [10-(4,5-dimethoxy-2-methyl-3,6-dioxo-1,4-cyclohexadien-1-yl)decyl]-triphenylphosphonium bromide (provided by Prof. Albin Hermetter, TU Graz, AT), diluted with PBS/1% DMSO in three different concentrations (200 nM, 300 nM and 400 nM) intraperitoneally with maximum administration volume 100 μl (controls receive 100 μl PBS/1% DMSO only). For injections, MitoQ is dissolved in PBS supplemented with sufficient DMSO (preferably 1%) to maintain solubility of antioxidants. Intraperitoneal or i.v. (tail vein) injections are given to pairs of mice and compared with vehicle-injected controls. These correspond to maximum tolerated dose of 20 mg of MitoQ/kg/day (750 nmol) according to Smith R. A. J et al., 2003, PNAS, 100 (9): 5407-5412.

On the second day MitoQ treatment is continued, additionally mice are intoxicated with 500 mg/kg paracetamol diluted with PBS po (maximum administration volume is 300 μl).

Controls receive 300 μPBS only. Vitamin E is given subcutaneously in the same intervals as MitoQ at doses of 40 to 100 mg/kg dissolved in corn oil/PBS (1:5). Cointoxication of carprofen with DDC (representing a model of liver disease condition) is performed to simulate the situation of drug-induced liver damage in patients receiving drugs which in other subjects do not cause hepatoxicity. To mimick this state carprofen is administered alone and in combination with the hepatoxic agent DDC: The animals are fed with DDC containing food (0.1%) and simultaneously receive 5 mg/kg of animal of carprofen (dissolved in 100 μl PBS/1% DMSO ip) for three days. Controls receive 0.1% DDC containing food or standard diet only. One group of carprofen-and DDC intoxicated mice receive daily 300 nmol and 400 nmol/animal of MitoQ (diluted with PBS/1% DMSO, ip). The corresponding control group receives intoxication and 100 μl PBS/1% DMSO without MitoQ.

Alternatively, MitoQ or MitoQ derivatives such as MitoS (a mixture of MitoQuinol [10-(3,6-dihydroxy-4,5-dimethoxy-2 methylphenyl)decyl]triphenylphosphonium methane sulfonate and MitoQuinone [10-(4,5-dimethoxy-2-methyl-3,6-dioxo-1,4-cyclohexadien-1-yl)decyl]-triphenylphosphonium methane sulfonate or MitoVit E can be supplemented to the diet. Doses are determined by measuring water or liquid diet consumption and mouse weight. Mice are fed in their drinking water for two days in the paracetamol- or three days in the carprofen and DDC intoxication without any gross signs of toxicity with 500 μM or 1 mM MitoQ or MitoS (maximum tolerated doses of 232 μmol/kg/day or 346 mmol/kg/day respectively, corresponding to 154 and 230 mg/kg/day for the 500 μM and 1 mM diets), or with 500 μM MitoVit E (a maximum tolerated dose of 105 gmol/kg/day corresponding to 60 mg of MitoVit E/kg/day) according to Smith R. A. J et al., 2003, PNAS, 100 (9): 5407-5412.

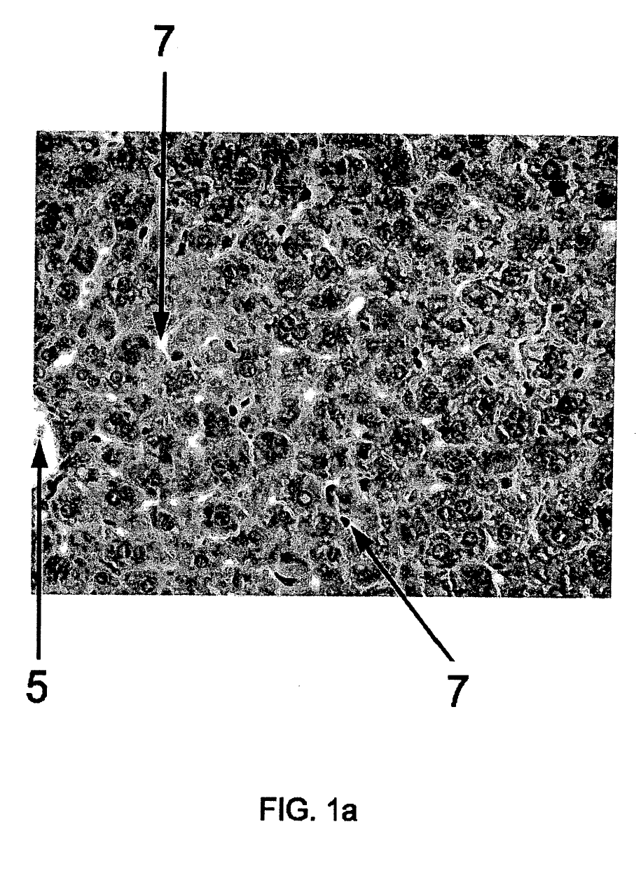

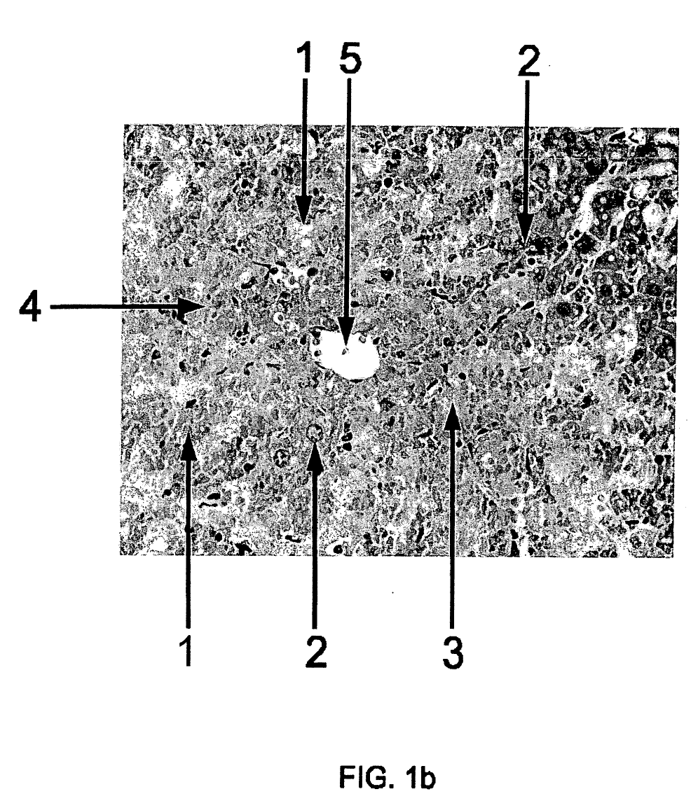

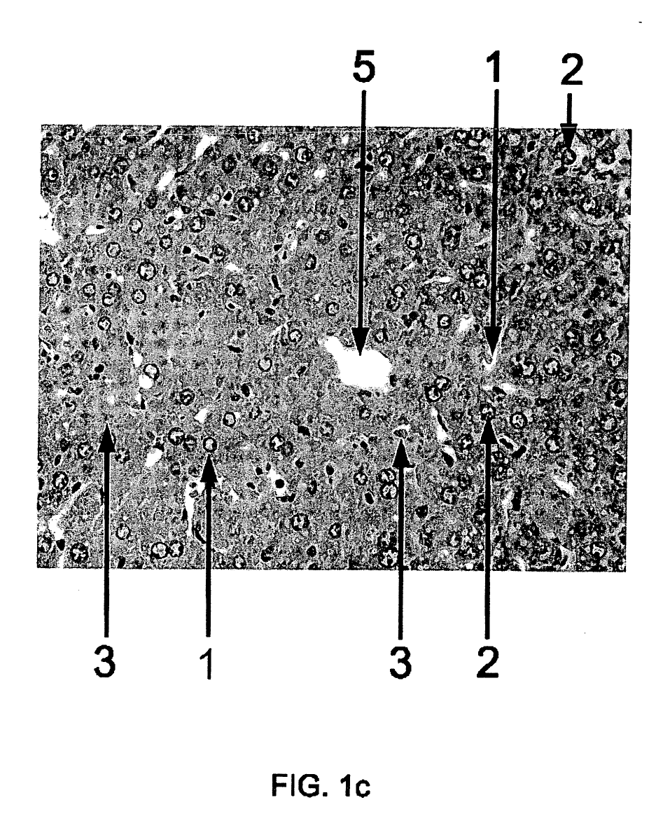

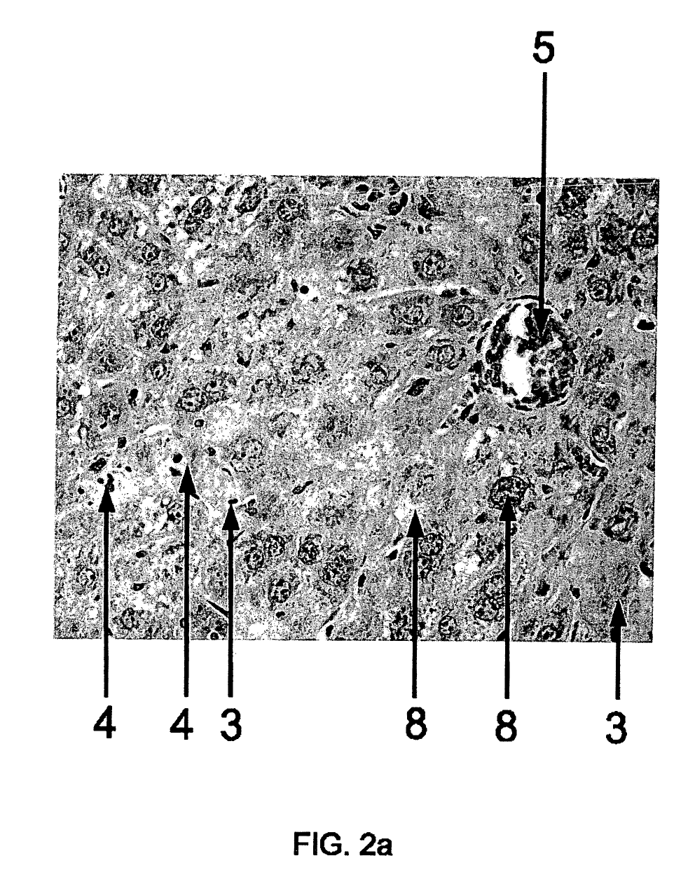

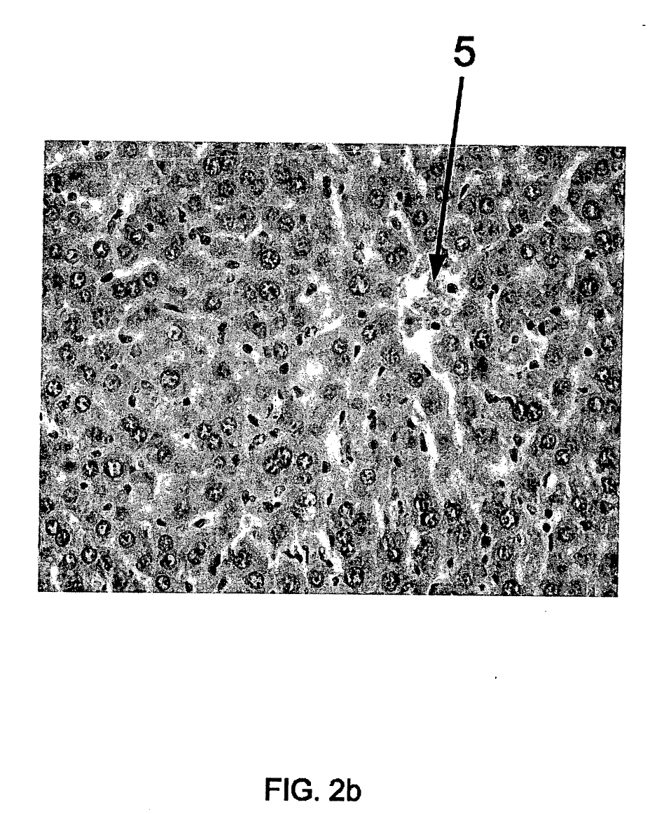

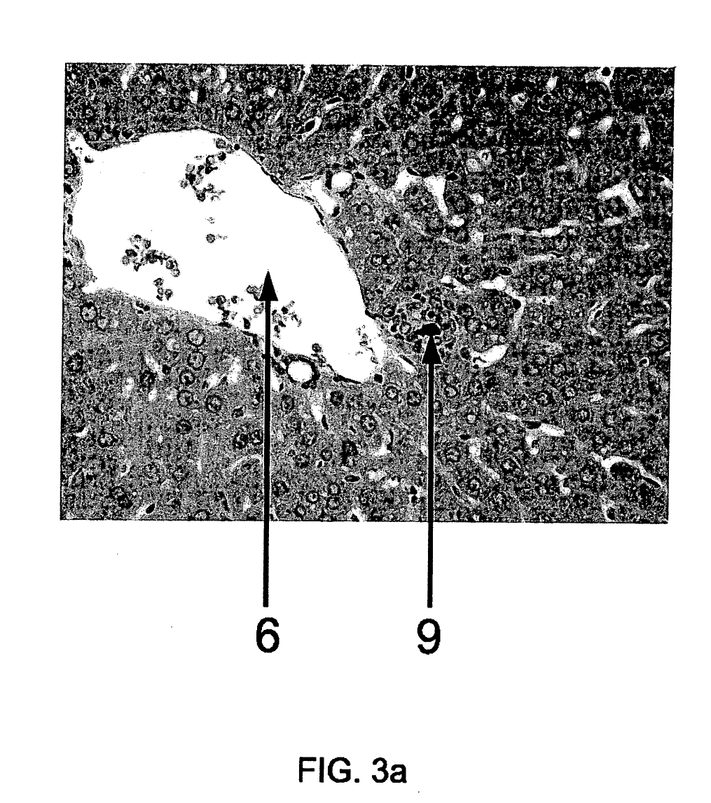

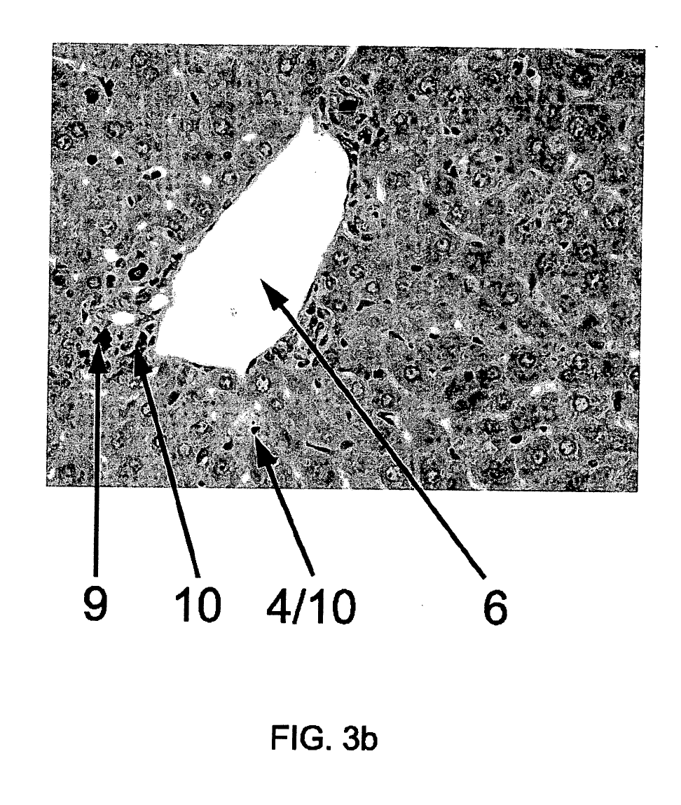

To practically assess in these short-term experiments the impact of mitochondrially targeted antioxidants according to the invention (e.g. MitoQ or MitoS) the presence (or absence) of inflammatory cells and the degree of hepatocyte damage such as necrosis (see FIG. 1a to 1c and FIGS. 2a and 3b and FIGS. 3a and 3b) are compared to the corresponding positive and negative controls.

Overall, under MitoQ or MitoS treatment the normal architecture represented by strands of hepatocytes bordered by sinusoids is again visible when compared to non targeted antioxidant (e.g. vitamin E) or untreated group of animals. The morphology of the hepatocytes and of the nuclei and structure of the cytoplasm is normal. Furthermore, the number of inflammatory cells is markedly reduced upon treatment with antioxidants according to the invention.

In long term experiments by using mice intoxicated for three weeks with valproic acid (0.71% weight/volume), the presence (or absence) of cell damage in liver samples of treated animals is determined and compared to the corresponding control groups of animals.

In one set of experiments, upon three weeks of intoxication with valproic acid, tested animals receive i.p. or i.v. (tail vein) injections of MitoQ or MitoS (1.25 mg/kg daily simultaneously given to pairs of mice), and compared with vehicle-injected controls and group treated with vitamin E.

Livers from tested mice in long term experiments are analysed by routine histology (standard haematoxylin/eosin staining according to Luna L. G., 1968, Manual of Histologic staining methods of the Armed Forces Institute of Pathology, 3rd edition. McGraw Hill, New York). The degree of cell damage is greatly reduced in the MitoQ treated animals intoxicated with valproic acid when compared to appropriate controls.

Overall, in both short and long term intoxication with paracetamol, carprofen in combination with DDC respectively, or valproic acid intoxication, the pronounced alterations of the liver are greatly ameliorated or reduced by the application of the antioxidants according to the invention used for the treatment or prophylaxis of drug-induced liver diseases

### Example 4

### Evaluation of Oxidative Stress Induced Proteins (Hemoxygenase I)

To evaluate hemoxygenase 1 (HO-1) protein expression know to be induced by oxidative stress (Suematsu M. and Ishimura Y., 2000, Hepatology, 31(1): 3-6) standard western blot analysis is performed using protein extracts derived from paracetamol- (or carprofen) intoxicated mice treated simultaneously for 1day (or 3 days) with MitoQ (diluted in 1% DMSO in PBS), vitamin E or just vehicle itself (see protocols in Example 3).

Liver tissues are resuspended in ice-cold RIPA-buffer (50 mM Tris-HCl pH 7.4, 250 mM NaCl, 0.1% SDS, 1% deoxycholate, 1% NP-40) supplemented with 2 μg/ml leupeptin, 2 μg/ml pepstatin, 2 μg/ml aprotinin, 1 mM phenylmethylsulfonylfluoride (PMSF), and 2 mM dithiothreitol followed by homogenization through sonication (2 bursts of 5 seconds) on ice. After incubation for 20 minutes on ice, the lysates are cleared by two centrifugational steps in a microcentrifuge at 13,000 rpm for 15 minutes at 4° C. and the supernatants are collected. Protein concentrations are determined by the Bradford assay (Biorad) using bovine serum albumin as a standard. Equal amounts of protein (typically 10-30 μg) are separated on a 12% SDS-PAGE gel and transferred electrophoretically to a polyvinylidene diflouride (PVDF) membrane (Hybond-P, Amersham) through Semidry-blotting (TE 70, Amersham). The membrane is blocked for 1 hour at room temperature in blocking solution [5% milk in TBS-T (25 mM Tris-HCl pH 7.4, 137 mM NaCl, 3 mM KCl, comprising 0.1% Tween-20)] and incubated with the primary antibody solution (prepared in TBS-T/1% milk) at 4° C. overnight with agitation. Antibodies specific for the following antigen is used: HO-1 (dilution 1:1000; Stress Gene) which cross reacts with constititively expressed iso-form HO-2 (36 kDa), and β-actin (1: 5000, Sigma), After removal of the primary antibody solution and several washes in TBS-T, the membrane is incubated with a HRP (horse-radish peroxidase)-conjugated secondary antibody (rabbit anti-mouse, 1: 1000; Dako) for one hour at room temperature. Following several washes in TBS-T, detection is performed through chemiluminiscence (ECL, Amersham) and exposing to x-ray film (FIG. 7). The intensities of the bands can be analysed densitometrically using Chemilmager 5500 software (Alpha Innotech) and each signal normalised to the intensity of the corresponding HO-2 showing significant reduction of HO-1 upon MitoQ treatment when compared to paracetamol intoxicated group of animals), and less significant reduction when compared to vitamin E treated mice (all protein signals normalized to 13-actin control). A marked decrease of paracetamol-induced overexpression of the hemoxygenase 1 under MitoQ treatment suggests that oxidative stress is greatly reduced by antioxidants according to the invention.

Furthermore, the protein expression level(s) of fatty acid binding protein (FABP) representing a sensitive marker for hepatocyte damage (Monbaliu D. et al., 2005, Transplant Proc. 37(1):413-416) is determined. Western blot analysis shows a significant decrease of FABP protein in paracetamol- or carprofen-intoxicated mice when compared to normal mice. Furthermore, under MitoQ treatment of drug-intoxicated animals FABP reaches almost control mice FABP protein expression values (controls represent non intoxicated group of animals treated with vehicle only), thus suggesting the effect of MitoQ in treatment or prophylaxis of diseases according the invention.

The amount of apoptotic cells in cryostat sections derived from drug-intoxicated mice treated with the antioxidants according to the invention can be semi quantified by anti caspase 3 immunohistochemical standard methods known in prior art (Brekken et al., 2003, The Journal of Clinical Investigation, 111, 4: 487-495) and compared to appropriate controls.

### Example 5

### Evaluation of the Effect of Antioxidants According to the Inventions on Blood Parameters

In serum from various tested animal groups following parameters indicating liver damage, namely bilirubin, alanine aminotransferase (ALT/GPT), aspartate aminotransferase (ASAT/GOT) and glutamate dehydrogenase (GLDH) are determined according to standard protocols in clinical diagnostics employing commercially available kits (No: 11552414; 11876805216; 11876848216; 11929992 all purchased by Roche A G, Switzerland) on a Hitachi/Roche 917 Analyser.

The reduction of serum liver enzymes in animals (as e.g. alanine- and aspartate aminotransferase treated with the compounds according to the invention indicates significant reduction of liver damage in MitoQ or MitoS treated samples when compared to vitamin E treated animals (less significant effect) and intoxicated control group, respectively, thus providing support for the therapeutic efficacy of these compounds in diseases according to the invention.

### Example 6

### Measurement of Reactive Oxygen Species (Ros) in Tissue Sections

To detect in situ generation of ROS in liver specimens derived from e.g. paracetamol- intoxicated and control tissues, fluorescence photomicroscopy with dihydroethidium (DHE, Molecular Probes) can be performed according to standard protocols (e.g. Brandes R. P. et al., 2002, Free Radic Biol Med.; 32 (11): 1116-1122). DHE is freely permeable to cells and in the presence of O₂ is oxidized to ethidium, where it is trapped in the nucleus by intercalating with the DNA. Ethidium is excited at 488 nm with an emission spectrum of 610 nm.

Liver samples are embedded in OTC Tissue Tek (Sakura Finetek Europe, Zoeterwonde, Netherlands) and frozen using liquid nitrogen-cooled isopentane. Samples are then cut into sections (5 μm-30 μm) and placed on glass slides. Dihydroethidium (5-20 μmol/L) is applied to each tissue section. The slides are subsequently incubated in a light-protected humidified chamber at 37° C. for 30 minutes and washed (2-3 times) with buffered saline solution (PBS) at 37° C. The sections are then to be coverslipped. The image of DHE is obtained by using fluorescence microscopy or laser scanning confocal imaging with a 585 nm long-pass filter.

Another approach well established in the art allows measuring the ROS production in drug intoxicated versus control liver tissue using a lucigenin chemiluminescence assay (Goerlach A. et al., 2000, Circ Res., 87(1): 26-32). Specimens of liver tissue are equilibrated in vials containing 1 ml of 50 mmol/L HEPES (pH 7.4), 135 mmol/L NaCl, 1 mmol/L CaCl₂, 1 mmol/L MgCl₂, 5 mmol/L KCl, 5.5 mmol/L glucose, and 5 μmol/L lucigenin as the electron acceptor. The light reaction between superoxide and lucigenin is detected using a chemiluminescence reader. The chemiluminescence signal is expressed as average counts per minute per mg dry tissue measured over a 15-30 min period. The chemiluminescent signal data are revealed after subtracting the background chemiluminescence observed in the absence of specimens.

This approach enables demonstration of the elevation of ROS in the liver derived from drug-intoxicated mice thus mimicking observations made in the patients suffering from the diseases according to the invention.

### Example 7

### The Effect of Antioxidants According to the Invention on Reduction of Reactive Oxygen Species (Oxidative Damage) in Mice Exposed to Paracetamol or Carprofen in Combination with Ddc

The general strategy of timelines and dosage regime(s) for drug (e.g. paracetamol or carprofen in combination with DDC) intoxication of tested animals and for their treatment with the antioxidants is identical to the experimental set-up used for determination of morphologic abnormalities (see Example 3).

The application of the antioxidants according to the invention, e.g. derivatives of vitamin E, coenzyme Q₁₀ or a glutathione peroxidase mimetic, provides a significant reduction of ROS levels in liver(s) exposed to drug inducing liver disease(s). This result further implicates impact of ROS in liver damage and demonstrates that this damage is mitigated by targeting e.g.MitoQ/MitoS to the mitochondria, a major cellular source of ROS. The reduction in the level of ROS measured with the methods according to Example 6 upon treatment with the targeted antioxidants indicates the therapeutic efficacy of these compounds for the diseases according to the invention.

### Example 8

### Measurement of Reactive Oxygen Species (Ros) in Immortalized Hepatocytes

Another simple set of experiments employing immortalized hepatocytes allows measurement of ROS production in these cells upon e.g. paracetamol intoxication.

To determine ROS production in the immortalized hepatocytes (e.g. by using Fa2N-4 or Ea1C-35 developed by MultiCell Technologies (U.S. Pat. No. 6,107,043)) intoxicated with effective concentrations of paracetamol (e.g. 10 mM for 2 hours), a standard experimental protocol according to Example 6 can be applied. Tested hepatocytes are grown in 96-well plates in culture medium (DMEM supplemented with 10% FCS, Gibco) to 80% confluency in the presence of paracetamol, subsequently washed with HBSS and incubated in the dark with DHE (10-50 μM) for 10 minutes at 37° C. Cells are then washed twice with Hank's balanced salt solution (HBSS, Gibco) to remove excess dye. Fluorescence is monitored in a fluorescence microscope (Olympus, Hamburg, Germany).

Alternatively, the generation of ROS can be measured by using the fluoroprobe 5-(and-6)-chloromethyl-2′,7′-dichlorodihydrofluorescein diacetate acetyl ester (CM-H₂DCFDA, Molecular Probes, Goettingen, Germany) which is converted to fluorescent dichlorofluo-rescien (DCF; Djordjevic T. et al., 2004, Antioxidants & Redox Signaling, 6: 713-720). To determineDCF fluorescence in a microplate reader (Tecan, Crailsheim, Germany), immortalized hepatocytes are grown in 96-well plates to 80% confluency in presence of paracetamol. The cells are then washed twice with Hank's balanced salt solution (HBSS, Gibco) and incubated in the dark with CM-H₂DCFDA (8.5 μM) dissolved in HBSS containing N-co-nitro-L-arginine methyl ester (L-NAME, 10 μM) for 10 minutes at 37° C. to prevent the formation of NO. After several washes with HBSS to remove excess dye, fluorescence is monitored by using 480 nm excitation and 540 nm emission wavelength. DCF fluorescence is standardized to the number of viable cells using the Alamar Blue test according to the manufacturer's instructions (Biosource, Nivelles, Belgium). Briefly, cells are incubated with Alamar Blue in phosphate-buffered saline (PBS), pH 7.4 at 37° C. to allow the indicator to change from the oxidized (blue) to the fully reduced (red) form. The absorbance is then measured at the wavelength of 580 nm.

Optionally, ROS production can be assessed by flow cytometric analysis of CM-H₂DCFDA stained cells. The cells intoxicated with paracetamol are detached and harvested by trypsinisation, collected by centrifugation and resuspended in HBSS at a concentration of 1×10⁶ cells/ml. Cells are then loaded with 8.5 μM CM-H₂DCFDA for 15 minutes in the dark at 37° C. before stimulation. The DCF fluorescence is monitored by analyzing 10,000 cells using 480 nm excitation and 540 nm emission wavelengths in a flow cytometer (Partec, Muenster, Germany).

The immortalized hepatocytes incubated for up to 72 hours in culture medium (DMEM and 10% FCS, Gibco) and supplemented with various concentrations of paracetamol, or DDC (EC₅₀=50 μg/ml of medium) or with up to 100 μM BSO can represent another suitable in vitro model mimicking observations made in patients suffering from the diseases according to the invention.

### Example 9

### The Effect of the Antioxidants According to the Invention on Reduction of Oxidative Damage in Immortalized Hepatocytes Intoxicated by Paracetamol

By employing standard protocols and following general strategy of time lines according to Example 8, the immortalized hepatocytes intoxicated with e.g. paracetamol (10 mM for 2 hours), DDC or BSO(ROS positive controls), respectively, and simultaneously treated with MitoQ/MitoS or vitamin E (in concentrations corresponding to EC₅₀=11M for MitoQ and EC₅₀=10 μM for vitamin E) provide a significant reduction in ROS formation.

In another experiment immortalized hepatocytes intoxicated by paracetamol or stimulated by e.g. 100 μM CoCl₂ (Sigma) can be used (Bel Aiba R. S. et al., 2004,. Biol. Chem. 385:249-57). Following the experimental set up described in Example 8, the drug-intoxicated immortalized hepatocytes are plated on a 96-well plate and serum starved for 16 h prior to the experiment. The hepatocytes are then washed once with HBSS (Hanks' Balanced Salt Solution, Gibco) and incubated with MitoQ in concentration range of 0.5 to 10 μM or the respective amount of DMSO (Sigma). After 15 min DCF is added to the cells (final concentration of 8 μM) and cells are incubated with the dye for 10 min. After loading the media is removed and fresh HBSS is added containing MitoQ and CoCl₂ (100 μM). The fluorescence can be measured in a plate-reader (Tecan Safire) after 0, 10, 20 and 30 minutes (Djordjevic T, et al., 2005, Free Radic Biol Med. 38:616-30).

Another approach allows measurement of the ROS production induced by paracetamol, (antimycin A or rotenone serving as positive controls) by using lucigenin chemiluminescence assay (experimental set up as in Example 8) in immortalized hepatocytes can be incubated in 6 well plates and stimulated by using paracetamol, or antimycin A in concentration 0-25 μM (preferably 0, 1 and 5 μM) simultaneously with or without MitoQ (or MitoS) in concentration range from 0 to 1000 nmol dissolved in DMEM (Gibco) for 3 hours at 37° C. After 3 subsequent washing the cells are equilibrated in plates containing 1 ml of 50 mmol/L HEPES (pH 7.4), 135 mmol/L NaCl, 1 mmol/L CaCl₂, 1 mmol/L MgCl₂, 5 mmol/L KCl, 5.5 mmol/L glucose, and 5 μmol/L lucigenin as the electron acceptor. The light reaction between superoxide and lucigenin is detected using a chemiluminescence reader (Lumistar, BMG laboratories, Germany). The chemiluminescence signal is expressed as average counts per minute and normalized to cell number as determined by cell counter (Casy Technology Instrument, Schärfe-System, Germany).

Overall, these experiments show a significant reduction in ROS formation thus further confirming a therapeutic benefit of mitochondrially targeted antioxidants in liver disorders according to the invention.

It will be apparent to those skilled in the art that various modifications can be made to the compositions and processes of this invention. Thus, it is intended that the present invention cover such modifications and variations, provided they come within the scope of the appended claims and their equivalents. All publications cited herein are incorporated in their entireties by reference

1. A method of treating patients with drug-induced liver diseases which comprises administering to the patient in need thereof a therapeutically effective amount of a mitochondrially targeted antioxidant compound comprising a lipophilic cation covalently coupled to an antioxidant moiety.

2. The method according to claim 1 wherein the drug-induced liver disease is a disease with pathological condition selected from the group consisting of acute liver damage, damage to liver blood vessels, chronic inflammatory nodules; chronic liver damage and hyperproliferation in the liver.

3. The method according to claim 2 wherein the acute liver damage is a dose-dependent or dose-independent liver damage.

4. The method according to claim 1 wherein the drug-induced liver disease is a disease selected from the group consisting of acute hepatitis, cholestatic jaundice, acute fatty infiltration of the liver, liver granulomas, chronic hepatitis, liver cirrhosis, liver fibrosis, chronic cholestasis and liver tumours.

5. The method according to claim 4 wherein the drug-induced liver disease is acute hepatitis or cholestatic jaundice.

6. The method according to claim 1 wherein the drug according to the invention is a drug from the group consisting of nonsteroidal antiinflamatory drugs, anticancer drugs, antibiotics, anticonvulsants, antiviral drugs, tuberculostatics, anesthetics, analgetics, antiarrythmics, antidepressiva, steroids, statins, insulin sensitizers, antihistamnica and herbal and dietary supplements.

7. The method according to claim 6 wherein the drug according to the invention is a nonsteroidal antiinflamatory drug or anticancer drug.

8. The method according to claim 1 wherein the liphophilic cation is the triphenylphosphonium cation.

9. The method according to claim 1 wherein the compound has the formula 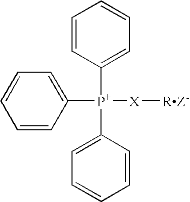
wherein X is a linking group, Z⁻ is an anion and R is an antioxidant moiety.

10. The method according to claim 9 wherein the antioxidant moiety R is a quinone or a quinol.

11. The method according to claim 10 wherein the compound has the formula

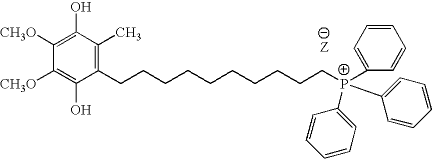

12. The method according to claim 9 wherein the antioxidant moiety R is a glutathione peroxidase mimetic.

13. The method according to claim 12 wherein the glutathione peroxidase mimetic moiety is

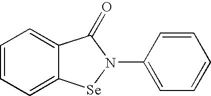

14. The method according to 9 wherein the anion Z⁻ is a pharmaceutically acceptable anion.

15. The method according to claim 14 wherein Z⁻ is halide.

16. The method according to claim 15 wherein Z⁻ is bromide.

17. The method according to claim 14 wherein Z⁻ is the anion of an alkane- or arylsulfonic acid.

18. The method according to claim 17 wherein Z⁻ is methanesulfonate.

19. The method according to claim 19 wherein the compound has the formula

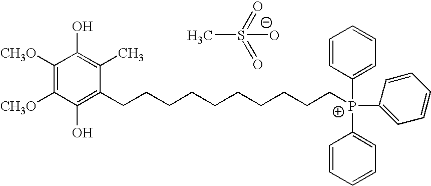

20. A method of treating patients as a prophylaxis against drug-induced liver diseases which comprises administering to the patient in need thereof a prophylactically effective amount of a mitochondrially targeted antioxidant compound comprising a lipophilic cation covalently coupled to an antioxidant moiety together or in sequence with the drug inducing a liver disease.

21. Use of a mitochondrially targeted antioxidant compound comprising a lipophilic cation covalently coupled to an antioxidant moiety for the treatment or prophylaxis of drug-induced liver disease.
